(19) United States
(12) Patent Application Publication
(10) Pub. No.: US 2015/0374334 A1
(43) Pub. Date: Dec. 31, 2015

(54) QUANTITATIVE TRANSMISSION ULTRASOUND IMAGING OF DENSE ANATOMICAL STRUCTURES

(71) Applicant: QT Ultrasound LLC, Salt Lake City, UT (US)

(72) Inventors: John C. Klock, Nicasio, CA (US); David T. Borup, Salt Lake City, UT (US); James W. Wiskin, Salt Lake City, UT (US)

(21) Appl. No.: 14/592,860

(22) Filed: Jan. 8, 2015

Related U.S. Application Data
(60) Provisional application No. 61/925,140, filed on Jan. 8, 2014.

(51) Int. Cl. A61B 8/00; A61B 8/14; A61B 8/08; A61B 8/15

(52) U.S. Cl. CPC A61B 8/4477; A61B 8/15; A61B 8/145; A61B 8/5207; A61B 8/5269; A61B 8/4461; A61B 8/4209; A61B 8/4494

(57) ABSTRACT

Methods and systems for imaging dense anatomical structures are provided. In one aspect, for example, a method for imaging a bone or a joint of a subject can include delivering a transmission ultrasound wave field from a transmission transducer array to a body part of a subject, receiving transmission data from the transmission ultrasound wave field at a transmission receiver array, delivering a reflection ultrasound wave field from a reflection transducer array to the body part of the subject, receiving reflection data from the reflection ultrasound wave field at a reflection receiver array, and generating an image of a bone or joint from at least one of the transmission data or the reflection data.

### PRIORITY DATA

This application claims the benefit of U.S. Provisional Patent Application Ser. No. 61/925,140, filed on Jan. 8, 2014, which is incorporated herein by reference.

### GOVERNMENT INTEREST

This invention was made with government support under The National Institute of Health Grant Nos. NCI 4R44CA110203-0022, 1R43CA123915-0, and 1RO1CA138536-01A2. The United States government has certain rights to this invention.

### BACKGROUND

Vertebrate animals have bone and joint systems that provide the ability to move about on land, in water, and in air. Vertebrate locomotory ability is facilitated by a framework of tissue structures that are similar for many higher vertebrate animals and humans. Bones, ligaments, tendons, muscles, nerves, blood vessels, and skin are all components of animal and human joints. These systems of bones, joints, and muscles develop early in embryonic life, and change as the animal or human matures into an adult.

A variety of conditions are known that may negatively affect bones and joints over the lifetime of a human or animal, examples of which can include developmental abnormalities, infections, inflammation, injury, benign and malignant tumors, and the like. Furthermore medical interventions (e.g., surgical procedures) can alter bones and joints both structurally and/or functionally. The surgical specialty of bone and joint manipulation is referred to as orthopedic surgery. In some cases, orthopedic surgery can play a role in diagnosing and treating various bone and joint conditions.

Medical imaging is useful in the management of medical conditions for humans and animals. Such imaging can provide visual and functional information, thus allowing a medical provider to determine if a patient has a medical condition. Detailed imaging of bones and joints can be performed using a variety of technologies, including X-rays to create images on film, computerized tomographic X-ray imaging using solid state detectors (X-ray CT), magnetic resonance imaging (MRI), and hand-held ultrasound imaging. Each of these methods has its own particular advantages, disadvantages and limitations. X-ray based imaging, for example, can be disadvantageous due to the use of ionizing radiation that can cause tissue damage with each use (in some cases imperceptible, in other cases the cumulative dose of radiation can harm the patient). MRI imaging requires exposure to high magnetic fields, as well as claustrophobia (disqualifying up to 25% of patients), long imaging times, and high cost of the technology. Hand-held ultrasound imaging can beneficial due to portability and safety due to the use of non-ionizing radiation, as well as being less expensive than X-ray, CT, or MRI imaging. Hand-held ultrasound does, however, have limited resolution, and the quality of the diagnostic procedure is operator-dependent, with the best results requiring more highly skilled operators.

### BRIEF DESCRIPTION OF THE DRAWINGS

For a fuller understanding of the nature and advantage of the present disclosure, reference is being made to the following detailed description of various embodiments and in connection with the accompanying drawings, in which:

FIG. 1 shows a scanning system according to one aspect of the present disclosure.

FIG. 2 shows a scanning system according to one aspect of the present disclosure.

FIG. 3 shows a scanning system according to one aspect of the present disclosure.

FIG. 4 shows a top-down view of an imaging chamber and coupler device according to one aspect of the present disclosure.

FIG. 5 shows an illustration of a coupler device according to one aspect of the present disclosure.

FIG. 6 shows a mobile imaging system according to one aspect of the present disclosure.

FIG. 7 shows a top-down view of an imaging chamber and transducer arrays according to one aspect of the present disclosure.

FIG. 8 shows a human knee imaged with the disclosed scanning technology, shown with the actual cut surface of the same human knee at the same level of the image, showing the synovium and cartilage in the coronal view according to one aspect of the present disclosure.

FIG. 9 shows a human knee imaged with the disclosed scanning technology, shown with the actual cut surface of the same human knee at the same level of the image showing the muscles and blood vessels in the coronal view according to one aspect of the present disclosure.

FIG. 10 shows a horse foreleg imaged with the disclosed scanning technology, showing the superficial and deep flexor tendons in the coronal view according to one aspect of the present disclosure.

FIG. 11 shows a horse foreleg imaged with the disclosed scanning technology, showing the superficial flexor tendon in the coronal, axial and sagittal views, reconstructed by the system according to one aspect of the present disclosure.

FIG. 12 is a horse foreleg imaged with the disclosed scanning technology, showing the superficial flexor tendon in the coronal view showing the sesamoid ligaments according to one aspect of the present disclosure.

FIG. 13 shows a flow diagram of a method of imaging dense anatomical structures according to one aspect of the present disclosure.

### DETAILED DESCRIPTION

Before the present disclosure is described herein, it is to be understood that this disclosure is not limited to the particular structures, process steps, or materials disclosed herein, but is extended to equivalents thereof as would be recognized by those ordinarily skilled in the relevant arts. It should also be understood that terminology employed herein is used for the purpose of describing particular embodiments only and is not intended to be limiting. Alterations and further modifications of the inventive features illustrated herein, and additional applications of the principles of the inventions as illustrated herein, which would occur to one skilled in the relevant art and having possession of this disclosure, are to be considered within the scope of the invention.

### DEFINITIONS

The following terminology will be used in accordance with the definitions set forth below.

It should be noted that, as used in this specification and the appended claims, the singular forms “a,” and, “the” include plural referents unless the context clearly dictates otherwise. Thus, for example, reference to “a transceiver” includes one or more of such transceivers and reference to “the logic processor” includes reference to one or more of such processors.

As used herein, “subject” refers to any mammal or other animal having at least bones and joints. Examples of subjects include humans, and may also include other animals such as horses, pigs, cattle, dogs, cats, rabbits, and aquatic mammals.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a numerical range endpoint by providing that a given value may be “a little above” or “a little below” the endpoint.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually.

This same principle applies to ranges reciting only one numerical value as a minimum or a maximum. Furthermore, such an interpretation should apply regardless of the breadth of the range or the characteristics being described.

The Disclosure

The present disclosure provides systems, devices, for performing ultrasound imaging of anatomical structures of a subject, including methods associated therewith. Such a system can be particularly useful for imaging denser anatomical structures, such as bone, joints, muscle, cartilage, and the like. In one aspect, quantitative transmission ultrasound (QTUS) can be utilized to image bones, joints, and other dense anatomical structures with great detail. In addition to increased soft tissue contrast and generally higher spatial resolution (e.g. 500 microns) as compared to other imaging modalities, QTUS allows for a high degree of imaging energy manipulation from, for example, a programmable beam former. Furthermore, QTUS can utilize a fixed, mechanical device, and as such, resulting images are highly reproducible. Because the speed of the ultrasound energy through the body part can be accurately measured, the QTUS scanner can also provide quantitative data for each area of the tissue that is imaged. QTUS technology is also inherently less expensive to manufacture than other imaging systems such as X-ray, CT, or MRI, and can thus more affordably deliver high-resolution medical imaging. QTUS is additionally safer to use than X-ray, CT, or MRI, because it functions using non-ionizing radiation and does not require the injection of contrast media. It is noted, however, that while not required for QTUS imaging, contrast media can be used in some aspects.

It has generally been understood by those skilled the art that ultrasound energy cannot penetrate bone and other more dense bodily structures. As a result, prior art reflection ultrasound imaging has been limited to soft tissues that are readily penetrable. Surprisingly, however, the inventors have discovered that ultrasound energy traversing through bone can be detected using inverse scattering techniques. High-resolution images of more dense anatomical structures, including bone and surrounding tissues, can therefore be obtained using QTUS as an exemplary imaging platform. Furthermore, the advantages of QTUS imaging over reflection ultrasound additionally include better soft tissue contrast and generally higher spatial resolution. QTUS imaging also provides for better quality 3D volume images, and is highly reproducible. Because the speed of the ultrasound energy through the body part is accurately measured, the scanner also provides quantitative data for each area of the tissue that is imaged.

The use of inverse scattering techniques makes it possible to more effectively utilize the sound energy traversing through bone, which can thus be utilized to images denser anatomical structures, including bones, joints, tendons, ligaments, and the like. However, due to the high impedance mismatch between bone and soft tissue, relatively little acoustic energy is able to penetrate into the bone and propagate through to the other side of the object being imaged to be received by the receiving transducer array. For an estimated bone impedance of 42 Rayl, the estimated signal propagating through would be decreased by ˜55 dB. There is, however, transmitted signal that misses the bone, or is mode-converted (e.g., from compressional to shear wave components) after partially propagating through the bone, that can be received at the receiver array. This mode conversion can be partially accounted for. The inventors have surprisingly discovered that in some aspects transmission wave calculations, when altered to incorporate shear wave moduli, can effectively model the wave energy conversion from compressional (P) wave to shear wave (SV and SH in the case of isotropic media, for example) and back again, thereby converting the weak transmitted signal into a usable signal for inverse scattering.

Furthermore, the two way algorithm can approximate the reflected wave with appropriate boundary conditions, and thus the reflected wave can be utilized in an inverse scattering algorithm as well. This can be particularly advantageous where ultrasound energy does not propagate through bone or other tissue very effectively, as noted above.

In a further aspect, the k-averaged algorithm partially accounts for large impedance tissues and thus can handle larger contrasts more effectively than the standard algorithm. As cancellous bone is porous, poromechanical theory can be utilized to account for and model wave propagation through porous media. One example of a useful poromechanic theory is the Biot theory of elastic material.

Furthermore, various image processing calculations according to present aspects of the disclosure include reflection algorithms, which have difficulty in the presence of bone. These reflection images can be processed to show detailed structures of denser anatomical structures, however. Once a speed of sound map has been constructed from transmission mode imaging, the refraction corrected reflection algorithm can be utilized. This reflection algorithm can also be implemented also without the refraction correction. In one incarnation the Lame parameters and X, are used, in another incarnation the full Stiffness matrix of elasticity is used, with or without suitable symmetries.

As an example, and without intending to be bound by any scientific theory, dense anatomical structures such as bones, joints, tendons, ligaments, and the like, can be imaged using speed map data derived from transmission mode imaging. Images having useful structure information can be generated from speed of sound data, attenuation data, and both speed of sound and attenuation data together. Additionally, detailed images are obtained by refraction correcting reflection imaging data using speed of sound data derived from transmission mode imaging data. Transmission energy (or transmission data) received after passing through the tissue by transmission mode imaging enables the creation of a speed map (or speed data) of the voxels within an image, or within the space to which the resulting data is mapped. Reflection energy (or reflection data) received after reflection by the tissue enables the creation of a reflection image, or reflection data useable to create a reflection image. By refraction correcting the reflection data, or in some cases a reflection image, dense anatomical structures can be shown in detail.

Additionally, while the speed of sound data can be used to correct for refraction, in some aspects the associated attenuation data can be used to create an automatically generated gain ramp for the reconstruction process. As such, in one aspect the reflection data can be refraction corrected using attenuation, or in other aspects, both speed and attenuation.

It is noted that any ultrasound signal or wave field design can be utilized that allows transmission mode imaging and reflection imaging, and any useful ultrasound signal characteristic design is considered to be within the present scope. In some aspects, however, short ultrasound pulses or “chirps” can be used in transmission mode imaging that contain, in some cases, multiple frequencies. In other aspects, large bandwidth pulses can be utilized for reflection mode imaging.

Various techniques are contemplated for refraction correcting the reflection data, and any such method or technique is considered to be within the present scope. In one aspect, for example, the refraction correction can be accomplished by modelling a beam (or a ray) that propagates according to an ordinary differential equation (ODE). As an example, the beam energy is followed along curved rays into the tissue, and the received signal energy can be mapped back into the tissue at the corrected location points. Additionally, the beam propagation can also be modeled as it actually occurs, i.e. as a wave front propagating through the heterogeneous tissue. This distinction can be relevant as the ‘beam’ so called is formed by time delays in neighboring elements, and the ‘beam’ is not in fact an infinitely thin ray. Also this ‘beam’ so called can be designed to be focused at successively further distances (dynamic focusing), but this focusing can be imperfect due to the very inhomogeneity in the tissue that causes refraction as well.

As such, in one aspect individual component data can be collected and the ‘beamforming’ process can be modeled. One exemplary method of accomplishing this modeling is performed using a ‘fast marching method’ for the solution of the partial differential equation (PDE) form of the eikonal equation. Note that there are two forms of the eikonal equation: the ODE form described above that is used to model idealized beams, and the PDE form that is a non-linear PDE that can be used to model the propagation of the wave front generated by the time delayed elements that surround the central element from which the ‘beam’ is assumed to propagate from.

As such, instead of modelling a single beam by the ordinary differential equation form of the eikonal equation, the wavefront as modeled by the partial differential equation form of the eikonal equation can be utilized. The ‘fast marching method’ is known in the literature, examples of which include “Shape Modeling with Front Propagation: A Level Set Approach”, Malladi, R., Sethian, J. A., and Vemuri, B. C. IEEE Trans; On Pattern Analysis and Machine Intelligence, 17, 2, pp. 158-175, 1995; and “a Fast Level set Method for Propagating Interfaces”, Adalsteinsson, D. and Sethian, J. A., J. Comp. Physics, 118, pp. 269-277, 1995, each of which is incorporated by reference in their entireties.

It is noted that, in some aspects, the reflection data can be collected from each component/element of the receiver array, that is, without using the beamforming capability. Such raw data can then be synthesized in software to dynamically focus at different depths, or synthetically focused to yield a reflection image.

It is additionally contemplated that shear wave acoustic energy can be used to image a body part. For example, one aspect a plurality of ultrasound frequencies can be sequentially or non-sequentially presented through the transducer array at different energies, such as, for example, from about 1 MHz to about 15 MHz. Such a procedure can facilitate ranges of penetration and higher resolution for the body part to be imaged. In some aspects, each of the ultrasound frequencies can each be presented at a range of different frequencies.

It is noted that, for any QTUS imaging procedure, in some aspects an imaging agent or agents can be used to enhance various aspects of the imaging procedure. Non-limiting examples of such contrast agents can include microbubbles, air-filled microcapsules, microparticles containing biological materials, antibodies, molecular probes, and the like. Contrast agents can be utilized as injectables, both at the imaging site and systemically, and in some cases as oral or topical applications. It is noted, however, that contrast agents are optional, and are not required for the generation of images of dense anatomical structures.

As has been described, the present disclosure provides devices, systems, and methods for imaging bones, joints, and other internal anatomic structures of a subject. In one aspect, QTUS protocol can be used to perform such imaging. In the imaging of a subject's limb, for example, bones, joint structures, ligaments, muscles, nerves, blood vessels, skin, tissue subcomponents, and other anatomical structures can be captured in high detail. It is noted that the present disclosure is not limited by the area or region of a subject being imaged, but the present scope includes any body part or anatomical structure capable of being imaged accordingly. Such a system can be a non-invasive, diagnostic tool to provide detailed information about the physiology (i.e. bulk tissue properties) and anatomy (i.e. physical architecture) of the joint. In one aspect, the system can be used as an adjunct to X-ray or MRI imaging to aid physicians, veterinarians, and other medially oriented professionals in diagnosing injury (e.g. orthopedic) by providing information about tissue properties that help to more clearly differentiate normal or benign from injured or otherwise affected tissue in the joint. In another aspect, the system can replace other diagnostic testing, such as diagnostic X-rays, MRI, hand-held ultrasound, and other imaging technologies currently used. In yet another aspect the system can be used to provide real-time imaging for medical procedures, such as, for example, invasive medical procedures.

In general, the systems according to aspects of the present disclosure can utilize ultrasound inverse scattering technology to produce a 3-D stack of tomography (2-D planar slice) images that can be similar or better in appearance and spatial resolution to CT or MR imaging methods. Direct 3-D imaging can be a further feature of the system. In one aspect, such images can be produced using two different techniques, namely Ultrasound Reflective Tomography (URT) and Ultrasound Inverse Scattering Tomography (UIST). Compared with conventional projection CT scans, URT images can be more detailed, easier to read, and are not obtained using potentially harmful ionizing radiation. Unlike conventional ultrasound, ultrasound images using inverse scattering technology completely penetrate and sample the entire body part, such as a joint for example, for increased uniformity and better overall resolution. In addition, such images are quantitative representations of ultrasound tissue properties, and therefore are not dependent on the system operator for image quality and consistency. The images can be reconstructed in three dimensions, thus providing an important visualization tool for diagnosis, biopsy, and surgery staging.

Numerous ultrasound imaging system designs are contemplated, and it should be understood that such designs can vary widely depending on the body part being imaged, the species of subject being imaged, the physical location of the imaging, permanent vs. mobile imaging systems, designer preferences, and the like. The present scope should not be seen as being limited by any system design, or the system designs shown below.

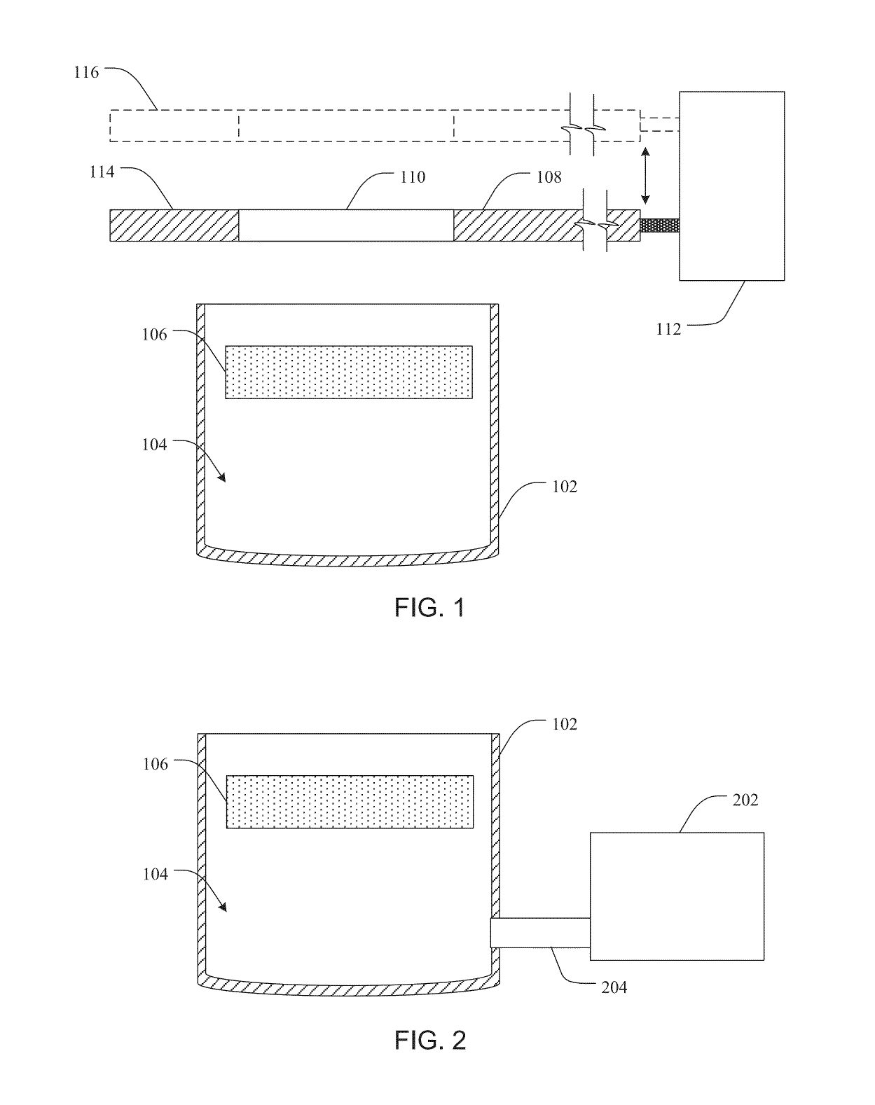

In one aspect, an ultrasound scanning system for imaging a body part of a subject is provided. In some aspects the body part can be a joint and/or a bone. Such a system can include an imaging chamber operable to contain a liquid transmission medium and to receive a bone or a joint of a subject into the transmission medium, and at least one transducer array disposed in the chamber and operable to transmit and receive ultrasound signals within the medium. In one exemplary embodiment, as is shown in FIG. 1, an imaging chamber 102 is shown having an interior 104 into which a bone or joint of a subject can be introduced (not shown). The interior 104 of the imaging chamber 102 can be filled with the transmission medium to facilitate the transmission of ultrasound therethrough. The transmission medium can be any liquid, including gels and jellies, or other material capable of deforming to receive a subject's body part therein that can facilitate the transmission of ultrasound through the chamber.

The imaging chamber can include a variety of configurations and be made from a variety of materials. It is noted that the imaging chamber structure and design should not be seen as limiting, and the details provided here are merely exemplary. In one aspect, however, the imaging chamber can be cylindrical, and in some aspects can be transparent. While the interior of the imaging chamber can be of any shape, a cylindrical design can be beneficial as it allows rotational motion of the arrays while minimizing volume. Transparent walls, while not necessary, allows the body part to be viewed during the scan, and allows an operator of the system to observe operation of the transducer arrays. Alternatively, the imaging chamber wall can be opaque or translucent, and in some aspects can have a window formed therein. The imaging chamber can also include fluidic devices forming inlet and/or outlet openings to allow fluid to enter and/or exit the bath. An upper end of the bath can be open to receive the body part, as described in greater detail below. It is noted that designs of imaging chambers lacking liquid transmission media is additionally contemplated.

The imaging chamber 102 can also include at least one transducer array 106 disposed therein. The transducer array 106 can be positioned to deliver ultrasound energy into the transmission medium, and to receive at least transmission and/or reflection energy from the transmission medium. In one aspect, a transmission array can include one or more receiver operable to receive ultrasound signals and one or more transceivers operable to transmit ultrasound signals incorporated into a given array. Thus receivers and transceivers can be mixed in the same array. In another aspect, receivers and transceivers can be divided into separate arrays. It is additionally noted that in some aspects a transducer can act as a transceiver and as a receiver. Furthermore, in some aspects the transducer arrays can include transceiver and receiver elements that are positioned to interact with one another to generate an image.

The transducer arrays can be configured to move within the imaging chamber. For example, the transducer array(s) can move within the chamber to obtain both reflection and transmission information used to generate images and diagnostic information. Such arrays can be designed to rotate, as well as move up and down to generate a complete 3-D data set for the area of interest or even for the entire body part. In general, ultrasound pulses can be used for two imaging modalities: reflective and transmissive. For reflective images, the system emits a pulse from one array and receives the reflected energy back in the same array. For transmissive images, the system emits a pulse from one array and receives the transmitted energy through the body part at a separate array. As one example, an array collecting reflective images can emit a pulse at a variety of positions around the body part. The number of incremental positions can vary depending on the design of the device and the imaging procedure, however in one non-limiting example, the array can emit pulsed at 20 positions (every 18 degrees) around the body part. As with the reflective image case, an array collecting transmissive images can emit a pulse at a variety of positions around the body part, and the number of incremental positions can vary depending on the design of the device and the imaging procedure. In one non-limiting example, however, during the same rotation sequence described for the reflective images, an array generating transmissive images can emit an ultrasound signal into and through the body part at 180 different locations (every 2 degrees) around the entire body part to be received by an opposing transmission receiver. This allows the system to simultaneously generate data for both reflection and transmission sound properties of the joint. Alternatively, the arrays can move and/or emit continuously, and in some aspects the reflective and transmissive arrays can generate images independent of one another, such as, for example, rotations that are exclusive for reflection or rotations that are exclusive for transmission.

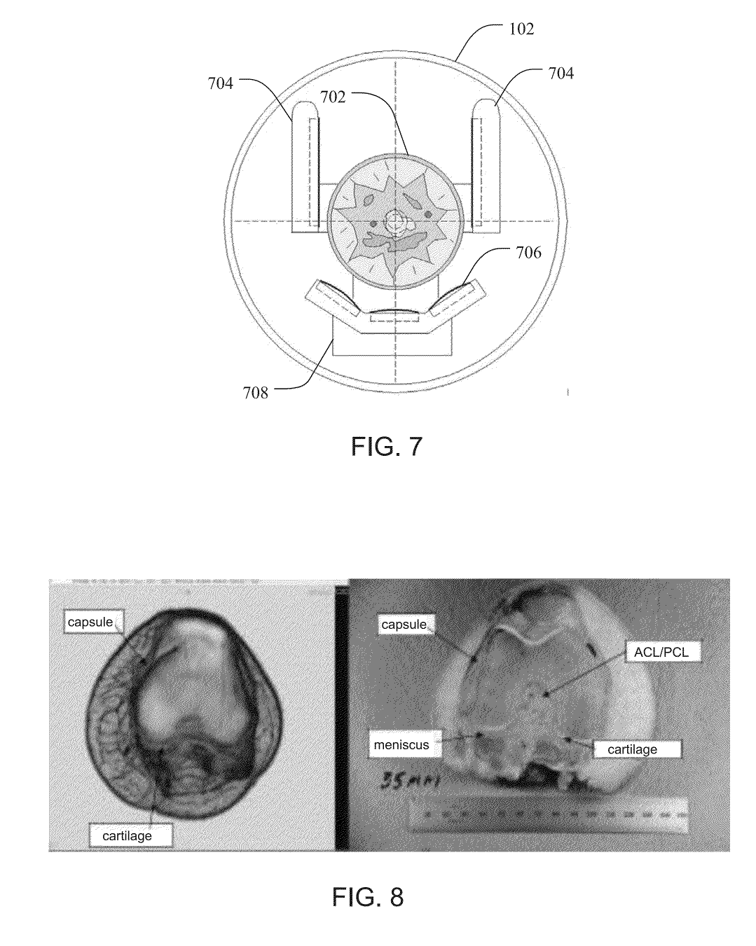

In one specific non-limiting aspect, a system can utilize at least two ultrasound arrays that rotate around the body part to generating true 3-D images and diagnostic information in a commercially viable timeframe, such as less than about 20 minutes per exam. The body part can be disposed in a bath of medium, such as liquid, water, or gel, or it can be placed into the fixed circular array as is described more fully below. The system can include two opposing ultrasound transducer arrays, as is shown in FIG. 7, movably disposed in the imaging chamber 102 to obtain both reflection and transmission information used to generate images and diagnostic information. The arrays are mechanically designed to rotate and move up and down generating a complete 3-D data set for the area of interest or even for the entire body part 702. In the case of FIG. 7, the system includes two opposing transmission arrays 704, and a reflection array 706 that can function as described above.

In some aspects, an imaging system can produce three separate images using two different imaging techniques: 1) transmission information generates images representing bulk tissue properties of speed of sound and attenuation of sound at each point in the body part; and 2) data generated from reflection information generates detailed reflective tomographic images that are refraction corrected. These imaging techniques are combined to effectively produce a three-dimensional stack of “slices” of the joint. Data from the ultrasound source is analyzed, and a quantitative map of tissue properties is rendered. In the “transmission mode” the energy propagates through the joint (or other soft tissue). In the “reflection mode”, the energy reflects back to the receivers. In both cases, the energy of the acoustic wave is refracted and scattered from the tissue it encounters. In this process multiple physical phenomena take place: reflection, refraction, diffraction, and multiple scattering events. These effects are generally ignored in present ultrasound, and as such, the image is significantly degraded, therefore rendering it useful only in differentiating architectural or structural properties within the body part. In present ultrasound imaging system it is impossible to acquire quantitative values at a level sufficient for diagnosis of tissue characteristics using standard reflection ultrasound or imaging. Further details of inverse scattering technology and imaging are disclosed in U.S. Pat. Nos. 4,662,222; 5,339,282; 6,005,916; 5,588,032; 6,587,540; 6,636,584; 7,570,742; 7,684,846; 7,699,713; 7,771,360; 7,841,982; 8,246,543; and 8,366,617, which are herein incorporated by reference in their entireties.

As one example configurations, transducer arrays can be disposed in the imaging chamber and carried by an armature, also disposable in the chamber. The armature can include a u-shaped member disposed on a vertical column that extends through a bottom of the imaging chamber. Each vertical arm of the u-shaped member can carry one of the arrays shown in FIG. 7. The u-shaped member can be sized to position the arrays around the body part. The arrays and can be rotatable around an axis of rotation, and displaceable vertically. For example, the armature can rotate around the vertical column, thus rotating the arrays. A rotational motor can be coupled to the armature to rotate the armature. For example, the rotational motor can be a rotational step motor coupled to the armature or vertical column by a belt. In addition, a linear motor can be coupled to the armature to linearly displace the armature, and thus the transducer arrays. For example, the vertical column can be carried by a platform on a plurality of rods. One of the rods can be threaded. The linear motor can engage the threaded rod such that rotation of the motor can raise and lower the platform, and thus the vertical column along with the rotational motor. A rotational and/or sliding seal can be formed between the imaging chamber and the armature, or vertical column, to seal the imaging chamber where the armature or vertical column passes through the bottom of the chamber. In addition, one or more bearings or rotational bearings can be disposed between the vertical column and the platform to facilitate rotation and reduce frictional forces. Thus, the platform can carry the armature and related motors to move the armature.

In addition, in some aspects the arrays can be tilted, or rotatable to have tilted orientation to allow imaging closer to an area of interest. For example, the arrays can be angled or directed in an upwardly angled direction so that the arrays emit upwardly at an angle and receive downwardly at an angle. Furthermore, the transducers can be disposed around the joint, and along the length of the joint, so that the transducers do not have to be moved or rotated.

In some aspects, transducer arrays can be incorporated into a fixed ring, as is described below (see FIG. 4). The ring can also be offset or non-concentric. The transducer arrays can send and receive ultrasound signals at a plurality of elevational locations along the body part, and at a plurality of rotational orientations around the body part at each elevational location. A linear motor can move the transducer arrays sequentially through a plurality of different elevational locations along the body part. Array elements can independently emit pulses at a number of distances from each other (depending on the number of elements in the circle) around the body part. During the same pulsing sequence, the transmitting elements can emit an ultrasound signal into and through the body part at many different locations (depending again on the density of array elements around the ring) around the entire body part. The resulting waveforms can be received by the opposing array elements. The arrays can then be moved to a different location along the body part and the sequence repeated. Alternatively, the arrays can emit during a continuous motion. Thus, the movement of the arrays and armature can be discrete, or stepwise through discrete position, or continuous. Additionally, the ring array can be tilted as described above.

It is additionally contemplated that a transducer array can be configured and sized to match a give subject and/or a given body part. Sizing the aperture of the array to the body part can result in greater comfort to the subject, and can allow a maximum amount of the joint to extend through the aperture and possibly better image quality.

Returning to FIG. 1, in another aspect the system can include a substantially horizontal table 108 disposable over the imaging chamber 102 and operable to support the subject thereon. It is noted that in some aspects a table or other support device for a subject can be oriented in any direction, from vertical to horizontal. An aperture 110 in the table can be positioned over the imaging chamber 102 such that a bone or joint of the subject can be passed therethrough and into the imaging chamber 102. A movement actuator 112 can be coupled to the table 108 to allow the table 108 and the imaging chamber 102 to be vertically displaceable with respect to one another. In some aspects, the movement actuator can be coupled, either directly or indirectly, to the imaging chamber. In yet another aspect, the movement actuator 112 can be operable to maintain the table over the imaging chamber in 1) a first position 114 whereby the table is sufficiently proximal to the imaging chamber to allow a bone or joint of the subject to be within the imaging chamber when the subject is supported by the table, and 2) a second position 116 whereby the table is sufficiently distant from the imaging chamber to allow a bone or joint of the subject to be suspended above the imaging chamber when the subject is supported by the table.

The system can also optionally include a securing device that is operable to secure the body part of the subject in the imaging chamber. Such a device can function to minimize movement of the subject during imaging, as well as providing comfort, such as, for example, a support rest that allows the subject to maintain a body position for a period of time. The securing device can be located at any position that is beneficial for the imaging procedure, and can vary depending on the design of the system and the body part of the subject. In some aspects the securing device can be located above the imaging chamber, such as, for example, coupled around a table aperture. In other aspects the securing device can be positioned within the interior of the imaging chamber, on top of the imaging chamber or otherwise affixed to the outside of the imaging chamber. In other aspects the securing device can include multiple securing sections that may or may not be coupled together in the same device. For example, a securing device may have a first securing section that is located below the transducer arrays in the imaging chamber and a second securing section that is located above the transducer arrays either in or above the imaging chamber.

In some aspects the system can include a variety of fluidic devices and/or components to process and handle the transmission medium. It is noted that the various components can vary depending on the design of the system and the nature of the transmission medium. In one aspect, as is shown in FIG. 2 for example, the system can include a conditioning chamber 202 fluidly coupled to the imaging chamber 102. The conditioning chamber can be used for a variety of conditioning tasks, such as thermal regulation of the transmission medium, cleaning of the transmission medium and/or the conditioning chamber, osmotic balance, liquid motion control, and the like, including appropriate combinations thereof. As such, the conditioning chamber can be utilized to condition the transmission medium before, during, and/or after an imaging procedure. Additionally, in some aspects conditioning or at least a portion of the conditioning can occur on the transmission medium in the conditioning chamber, while in other aspects the conditioning or at least a portion of the conditioning can occur on the transmission medium in the imaging chamber. The fluidic coupling between the imaging chamber 102 and the conditioning chamber 202 can be any fluidic coupling mechanism known. In one aspect, the system can include a fluidic system 204 operable to transfer the transmission medium from the conditioning chamber 202 to the imaging chamber. In some aspects the fluidic system 204 can also operate to transfer the transmission medium either back into the conditioning chamber or to another chamber or to a waste or recycling system.

As such, a portion of the body of a subject can be place into the imaging chamber for an imaging procedure. The imaging chamber can then be filled with the transmission medium, either prior to, during, or following the introduction of the body part into the chamber. The transmission medium is operable to transmit ultrasound energy. Because the transmission medium is also coupled to the at least one transducer array and the body part, ultrasound energy is efficiently transmitted therebetween. It is noted for this and subsequent figures, the use of callout numbers used in previous figures denote the same or similar element of that previous figure, and that the description of that element should be incorporated into the present figure where appropriate.

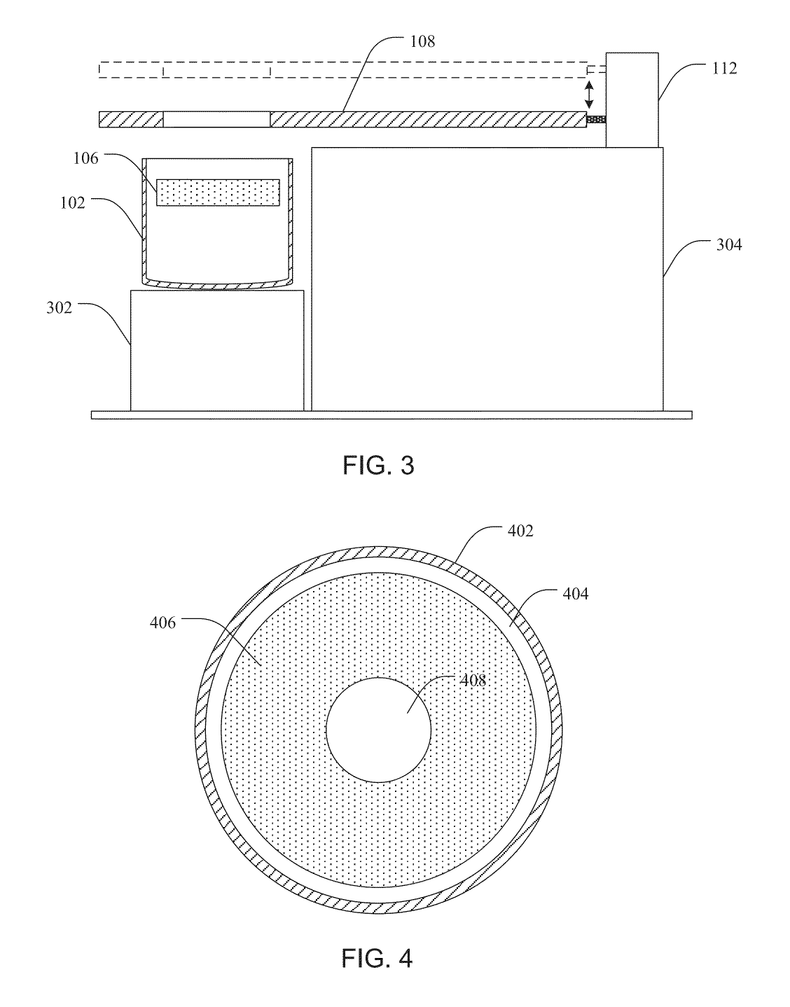

In another aspect, as is shown in FIG. 3, the system can include an imaging chamber support 302 for supporting the imaging chamber 102. The imaging chamber support 302 can be operable to perform a variety of functions in addition to merely supporting the imaging chamber 102. Non-limiting examples include 1D, 2D, 3D, and/or rotational movement of the imaging chamber 102, 1D, 2D, 3D, and/or rotational movement of the at least one transducer array, communication coupling between the at least one transducer array and any computer or other electronic device sending or receiving data or other signals from the transducer array, thermal regulation of the imaging chamber 102 and/or the transmission medium, and the like, including appropriate combinations thereof.

Furthermore, in some aspects the system can include a housing 304 capable of providing support and substance to the system, as well as containing various system elements. For example, in one aspect the housing 304 can provide support to the movement actuator 112 and the table 108. The housing 304 can be continuous or discontinuous with the imaging chamber support 302. Furthermore, in some aspects various ultrasound generation and/or ultrasound analysis systems, including various communication networks associated therewith, can be incorporated either fully or partially into the housing 304, and in some cases into the image chamber support 302.

In some aspects, the system can also include a light source (e.g., a laser pointer) to project a light beam (such as a fan beam) onto the body part at an area of interest. The area of interest can be marked prior to immersing the joint into the bath or into the ring array. The area of interest can be determined beforehand by reference to body part examinations, X-rays, etc. The light source can be mounted to the armature, and can be positioned at the arrays. Thus, the armature and arrays can be raised or lowered until the light beam from the laser pointer aligns with the mark on the body part corresponding to the area of interest. This position can be saved in the system as a center of the area of interest, and the scan can begin and end at a predetermined distance above and below the center of the area of interest. It will be appreciated that the position of the armature, and thus the arrays, can be determined from the motors used to position the armature, or from other sensors.

In addition, a camera can be positioned to provide an image of the body part and the arrays. The camera can be coupled to the system and/or a display or control module associated with the system. The camera can be mounted on the armature or ring array and positioned thereby. A horizontal line, or cross-hair, can be provided on the display, camera, or system to align the camera, and thus the arrays, with the mark on the body part corresponding to the area of interest. The camera can also include a light source, such as, for example, one or more lights, LEDs, or the like.

The various systems, devices, and methods of the present disclosure can also utilize an ultrasound transmission coupler in lieu or in addition to a liquid transmission medium where appropriate. For example, a sleeve or other configuration of coupler can be positioned around or in contact with a region of the subject to be imaged. In other words, the coupler can surround the region of the subject in some cases, and the coupler can contact only a portion of the region in other cases. The coupler thus facilitates the transmission of ultrasound energy between the transducer array and the subject, and as such the design and placement of the coupler can vary depending on the desired use. In some aspects a liquid, semiliquid, gel, or other like substance can be introduced between the transducer array and the coupler and/or between the coupler and the subject to improve ultrasound transmission therebetween. Additionally, in some aspects the coupler device can have a fixed shape and material composition, while in other aspects the coupler device can be a reservoir or can contain a reservoir that can be filled with a transmission medium that can transmit ultrasound. Such a fillable coupler device can conform to the shape of the body part, and thus may improve ultrasound transmission.

Accordingly, in one aspect an ultrasound scanning system for imaging a bone, joint, or other anatomy of a subject can include an imaging chamber operable to receive and contact a coupler device engaging a region of a subject, and at least one transducer array. In one aspect the at least one array of transducers can be positioned to at least partially encircle the coupler device. As such, a portion of the body of a subject is coupled to a coupler device that is operable to transmit ultrasound energy. Because the coupler device is coupled between the at least one transducer array and the body part, ultrasound energy is efficiently transmitted therebetween. The coupler device can surround the body part, a portion of the body part, or the coupler device can partially surround the body part or portion of the body part. The coupler device can also merely contact a portion of the body part sufficient to transmit ultrasound between the transducer array and the body part. Additionally, in some aspects the transducer array can be a ring of transducer arrays configured to couple to and/or to surround the coupler device.

In one aspect, as shown in FIG. 4, a system can include an imaging chamber 402 and at least one transducer array 404 positioned within or otherwise associated with the imaging chamber 402. FIG. 4 shows a circular transducer array, however it is contemplated that the imaging chamber can include multiple transducer arrays and/or non-circular transducer arrays. A coupler device 406 is positioned within the imaging chamber 402 to facilitate transmission of ultrasound energy between the transducer array 404 and the body part of the subject that is positioned within a receiving cavity 408 of the coupler device 406.

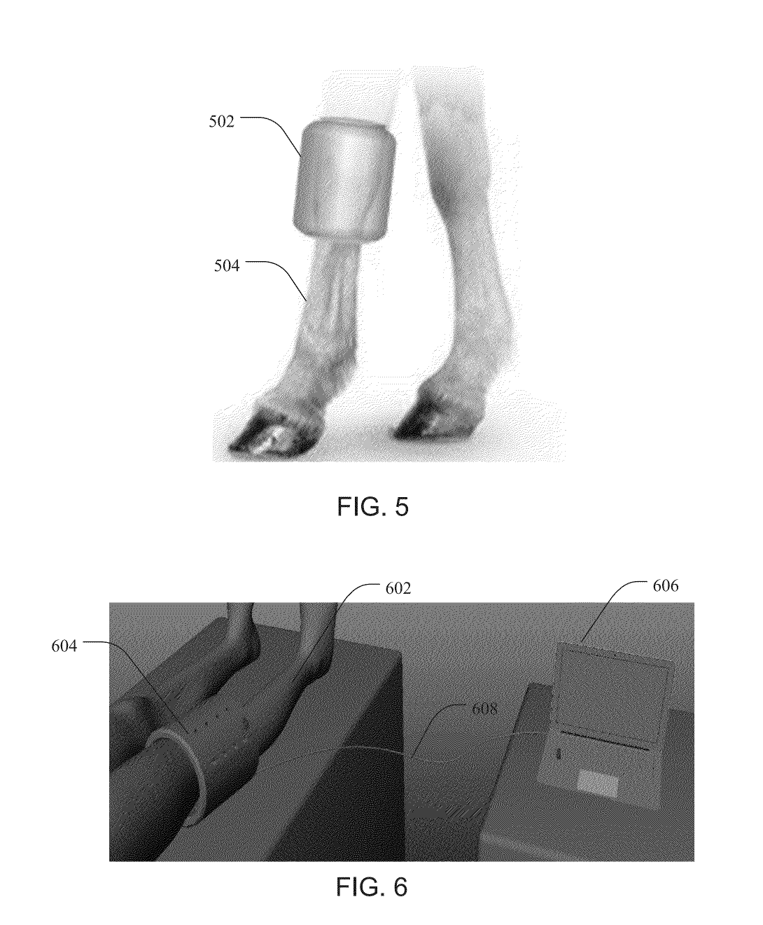

Turning to FIG. 5, a coupler device 502 is shown encircling a leg 504 of a horse, and particularly a joint in the leg of the horse. Such a sleeve-like coupler can thus be positioned around the leg to facilitate ultrasound transmission from many directions. In one aspect the coupler device 502 with the leg 504 can be placed into an imaging chamber of an imaging system. In other aspects, transducer arrays can be positioned around the coupler device or incorporated into the coupler device in order to provide a more mobile imaging solution.

One example of such a mobile system is shown in FIG. 6. In this case, the knee of a human leg 602 is being imaged by the system. A housing 604 including the transducer array or arrays (not shown) is shown coupled to the knee of the leg 602. A coupler device (not shown, obscured by housing) is positioned between the knee and the transducer array. The system can also include a computer 606 or other electronic device or system to control the transducer array and/or to gather data from the imaging procedure. The computer 606 can be functionally coupled to the transducer array by a variety of communication pathways. Non-limiting examples can include electrical wiring, fiber optics, Bluetooth, wireless networking, infrared communication, and the like, including appropriate combinations thereof. FIG. 6 shows an electrical wired connection 608 between the computer 606 and the transducer array in the housing 604. In some cases such a mobile scanner could be used as a safe and inexpensive “point of care” technology. While such technology can be useful in any environment, one potential use would be in economically-challenged areas of the world.

Turning to QTUS imaging in general, it should be understood that numerous system configurations of the ultrasound generation, delivery, capture, and analysis are contemplated, and that any such configuration is considered to be within the present scope. In one aspect, for example, an architecture or platform can include custom boards to provide data rates fast enough to store and process the amount of data gathered by the arrays. The architecture of the boards can vary depending on the design, provided the boards can operate at sufficiently fast data rates.

To render the images of the body part, there is a complex series of image processing steps that can be used. One practical method uses frequency domain data and a parabolic approximation to the full Helmholtz equation. This yields a very fast forward problem solver, the concomitant Jacobian is straightforward, and the desired (see below) fast implementation of the adjoint of the Jacobian calculation can be carried out after some algebraic manipulation, see (Wiskin, Borup, Johnson, & Berggren, 2012, entirely incorporated herein by reference). These algorithms are described in greater detail in U.S. Pat. Nos. 5,588,032; 6,005,916; 6,587,540; 6,636,584; 7,570,742; 7,684,846; 7,841,982; and 8,246,543 which are incorporated herein by reference in there entireties.

Other implementations of parabolic approximation can additionally be utilized, for example those that utilize an averaged background speed of sound, two-way propagating fields, or the like.

The following description is one exemplary embodiment of a hardware/software system that can be utilized to gather and process data from the image scanner. In some aspects, components of the platform can be used to control the ultrasound delivery and the mechanical manipulation of the transducer arrays. It is noted that as the technological level of computer systems increase over time, it is understood that the architecture of the present hardware/software platform can change as well. The present scope is not limited by the current state of computer technology, and it is understood that advances in computer technologies are considered to be within the present scope.

In one non-limiting example, the architecture of a useful hardware/software platform can resemble a Linux cluster super-computer. The cluster can include five or more nodes interconnected at high speed, such as by, for example, a high-speed Ethernet network. In addition, each node can have access to a shared file system. Two or more of the nodes can be configured as Compute Nodes. Each Compute Node can include a Single Board Computer (SBC) and a Fibre Channel Host Adapter (FCHA). Two of the nodes can be configured as data acquisition nodes. Each Data Acquisition Node can include a SBC, FCHA, Waveform Generator Card, Data Acquisition Cards, and Mux Cards. The FCHA can connect the SBC to the shared file system.

An exemplary Compute Node can include a SBC, FCHA and CompactPCI backplane that the two cards plug into. One or more of the backplanes can be installed in a 19 inch rackmount chassis. The chassis provides power and cooling to the cards plugged into the backplanes.

The design and components of an SBC can vary depending on the computational level of a given imaging process and the desired speed of data processing. Generally each SBC can include a CPU, volatile memory (e.g., 16 GB of SDRAM), an interface to the Compact PCI backplane, and an onboard PCI bus. The PCI bus can support any number of peripherals, depending on the design of the system and the preferences of the designer and/or operator. Non-limiting examples of such peripheral/peripheral devices can include mice, keyboards, parallel ports, data storage interfaces, USB, networking, and video controllers. The data storage interface can include any include any type of interface, including, for example, Integrated Drive Electronics (IDE), Enhanced IDE (EIDE), ATA, Serial ATA (SATA), and the like. Non-limiting examples of networking interfaces include wireless cards for Wi-Fi networking, Bluetooth interfaces, 10/100/1000 Ethernet cards, fiber optic communication, and the like. Video controllers can vary widely, and can include any video card that can process at a desired level given the image processing operations. As such, in one aspect the video card or cards can be PCI based, and can interface with the PCI bus as described. In other aspects the SBC can include a dedicated video card bus such as, for example, PCI Express, Accelerated Graphics Port (AGP), and the like. In some aspects, the SBC can include multiple video processing cards. Additionally, in other aspects the SBC can include onboard video processing.

As with many computer components, CPUs can vary widely and are constantly increasing in computational power over time. The CPU used in the SBC can vary across image scanning and processing systems, and can also vary within a given system. For example, a system used in medical diagnostic procedures may utilized all high end CPUs in order to process images rapidly to facilitate diagnosis. Also, in situations where the scanning system is used to image a joint or other body part in real-time during a medical procedure, extremely fast processing may be critical. In situations where processing speed may be less important, slower more economical CPUs may be acceptable. Additionally, in some aspects a given system can have different CPUs in different SBCs. Different SBCs in a system may have different processing needs and can, therefore, utilized different CPUs. This is similarly true for other components of the SBCs. In other aspects, all SBCs in a system can utilize the same or similar CPUs regardless of differential SBC processing. Non-limiting examples of current CPUs includes Pentium processors, Core i3, i5 and i7 processors, Xeon processors, AMD FX, A-Series, Phenom II, and Athlon II processors, and the like.

Additionally, in other aspects it is contemplated that computational tasks can be accomplished using one or more GPUs. For example, in one aspect the processing of the transmission data from an image scanning procedure can be performed on a GPU device or GPU technology. In another aspect, the processing of the reflection data from an image scanning procedure can be performed on a GPU device. In other aspects, both the transmission and reflection processing can be performed on one or more GPU devices. As such it is considered to be within the present scope to perform any portion of the computation and data processing that can be processed via GPU technology on a GPU device. Moving such computation from CPU processing to GPU processing can dramatically increase the speed of intensive calculations, such as the transmission and reflection calculations, in some cases by a factor of 5, 10 or more. Accordingly, the above described node configurations can additionally include GPU devices where appropriate.

Regarding the GPU device hardware, it is contemplated that any GPU device that is capable of such data processing is considered to be within the present scope. Non-limiting examples of off the shelf GPU devices can include NVidia Tesla K10, K20, K20X, K40, and the like. GPUs can be programed by various programing platforms, including, for example, NVidia's CUDA technology. It is also contemplated that the GPU technology is not limiting, and that future generations of GPUs are considered to be within the present scope.

An exemplary Data Acquisition Node can include the SBC, FCHA, Data Acquisition (DA) cards, Mux cards, Waveform Generator (WFG) card, the CompactPCI backplane that the cards plug into and a 19-inch rack mount chassis. The chassis provides power and cooling to the backplane and also provides a high-voltage power source for the Mux cards. One of the Data Acquisition Nodes can be designated as a master. The Master Data Acquisition Node can include a Motion Control card in addition to the cards found in the Data Acquisition Node. The Motion Control card can be used to control the motion of the transducer array(s).

The Mux card is the interface between the transducer array and the DA Card. A high density coax cable assembly can connect the Mux card to the transducer array, although other comparable connections are contemplated. One example of a Mux card can accept 256 analog inputs/outputs to/from the transducer array. Through inputs for the DA Card, the Mux card can select 16 of the 256 channels for amplification (70 db) and filtering. The 16 channels of conditioned analog data are then presented to the DA card via the CompactPCI backplane. One exemplary DA card has 16 14-bit A/D chips. Each A/D chip digitizes the analog data received from the Mux card at a rate of 33 million samples per second. Each A/D chip stores the converted data in a double-buffered First-In-First-Out (FIFO) for later storage in the SBC's memory or directly to the Fibre Channel Host Adapter.

The shared file system can be any data storage system capable of storing data from the various nodes of the system. Such can include any non-transitory computer readable media system having sufficient capacity and speed to be operational with the system. Non-limiting examples can include banks of disk drives, RAID arrays, cloud storage, and the like.

The architecture is configured to funnel data rapidly to the shared file system. The shared file system is available to store data and make data available for processing or other output use. As one example data can be processed from a first slice, or data from the first slice can be accessed for computation, while storing data from the second slice. The present architecture thus provides for efficient and flexible examination of a subject. In a medical setting for example, a technician can review the data or generated images while the system is scanning. In addition, the data or images can be provided to a physician immediately after an examination to quickly provide results to the subject. Furthermore, in many cases the present architecture can reduce the size and cost of the computing components.

The system can also include various other components, including for example, a power source, a high voltage source (e.g., for pumps and the like), a low voltage source (e.g., for sensors, control valves, and the like), computer(s), and drive array(s), etc. Many or all of the various components can be contained in a housing so that the system can be compact and self-contained. A terminal can be coupled to the computers and/or other components in the base by standard connections. The terminal can be remote from the base, or can be physically coupled thereto.

In some aspects, the platform can include a Waveform Generator (WFG) to generate a digitally-programmed waveform for use by the system. In one aspect, the WFG can function as the source of the signal used to excite the ultrasound transmission transducer array.

In some aspects, the platform can also include a beam former. The beam former can be used to generate a digitally-programmed waveform for use by the reflection system. In addition, the beam former can create a time variable gain control to vary the gain of the transducer array amplifiers. The Beam Former can also generate master timing signals used to synchronize the data acquisition subsystem. One example of a suitable beam former is the cQuest Cicada Subsystem by Cephasonics Corp.

In one specific aspect, a QTUS scanning system having a rotating transducer array is provided. As is shown in FIG. 7, The system can include an imaging chamber 102 and a tri-arm 708 upon which two opposing ultrasound transducer arrays 704 and a reflection array 706 are mounted to allow the arrays to move in relation to a body part 702. The arrays rotate and move up and down with the tri-arm to generate a complete 3-D data set for the area of interest or even for the entire body part 702. The transmission data is multiplexed to DA cards, and transferred to an on board RAID, where a separate processor (GPU card enhanced) applies the inverse scattering algorithm, then uses the resulting speed of sound (SOS) and attenuation map in the refraction corrected algorithm discussed herein. The reflection data is acquired through an independent DA system that also beam-forms the data.

For further details of systems that can be utilized to at least partially implement the QTUS imaging technology described herein, see U.S. Pat. No. 8,366,617, which is incorporated herein by reference in its entirety.

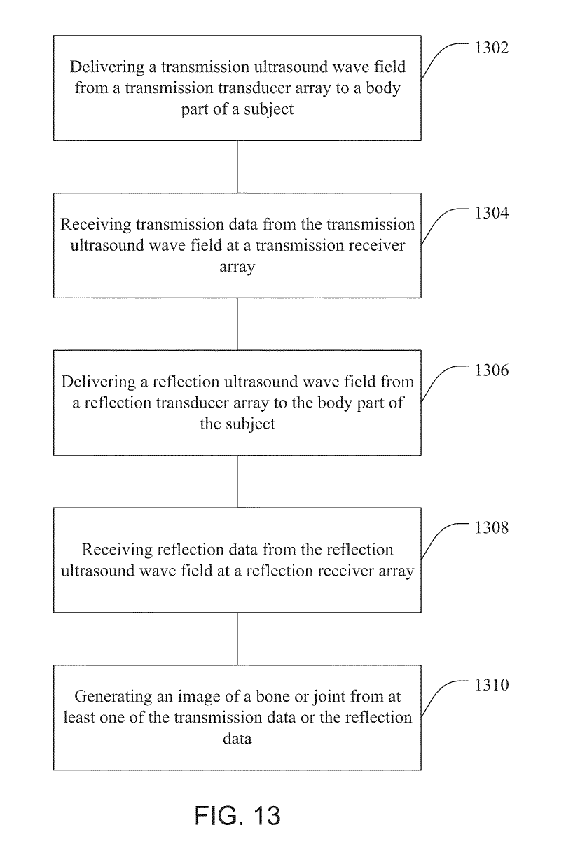

It is additionally contemplated that various systems including software platforms can be implemented to provide varying degrees of functionality to the QTUS system. Such software platforms and associated architectures should not be seen as limiting, and other implementations that differ from the following are considered to be within the present scope. In some aspects a system can be implemented to merely drive the ultrasound imaging apparatus. In other aspects, a system can drive the ultrasound imaging apparatus and/or process and analyze data to derive QTUS image results. In other aspect, systems can be realized that integrate the QTUS imaging system with higher level information processing such as, for example, medical records at a medical facility. One example of a software system including such integration is shown in FIG. 13.

Regarding the creation of transmission images in general, further details can be found in U.S. Pat. No. 8,246,543, which is incorporated herein by reference in its entirety. While the creation of reflection images is described herein and is contained in various patent references described above, the following pseudo-code provides an extremely high level summary of one such non-limiting process:

```
For lview = 1 , nview do
  If (correct_refraction = ‘yes’) read in filtered speed image
  Otherwise set slowness(I,j,k) = 1. For all I,j,k
  Apply digital gain (read in support)
    Or set support identically = 0.
    For lprobe = 1,3 do
      For level = 1,nlevels do
        Read in data
        For each beam: j=1,nbeams
          Compute Hilbert Transform → form analytic signal
        Enddo (beams)
        For each beam: j=1,nbeams; do
          Compute ray path through breast
          Apply digital gain appropriately
          Interpolate energy to reflection grid (3D) based on
          ray path (solution to eikonal equation)
        Enddo (beam)
      Enddo (level)
    Enddo (probe)
Enddo (view)
Store images by view
Blend images to get 360 degree compounding or ‘clock view’ images.
Post-process 360 compounded volume and/or clock view images.
```

The system or arrays can perform an initially, more rapid (or less detailed) scan of a larger length of the body part. Such an initial scan can be used to identify the area of interest in the body part for further scanning. Then, the system can perform a subsequent, slower or more detailed scan of a smaller length of the body part around the area of interest. For example, a leg can be scanned rapidly to locate an area of interest in the knee joint, after which the knee joint is scanned more slowly at higher resolution. Similarly, a joint such as a knee joint can be rapidly scanned to locate an area or structure of interest within the knee joint, after which the area or structure of interest can be scanned more slowly at higher resolution.

The present disclosure additionally provides methods for imaging dense anatomical structures of a subject, including bones and joints. In one specific aspect, a method for imaging a bone or a joint of a subject is provided, as is shown in FIG. 13. Such a method can include 1302 delivering a transmission ultrasound wave field from a transmission transducer array to a body part of a subject, 1304 receiving transmission data from the transmission ultrasound wave field at a transmission receiver array, 1306 delivering a reflection ultrasound wave field from a reflection transducer array to the body part of the subject, 1308 receiving reflection data from the reflection ultrasound wave field at a reflection receiver array, and 1310 generating an image of a bone or joint from at least one of the transmission data or the reflection data.

In one exemplary embodiment of the present disclosure, a method for imaging a bone or a joint of a subject comprises:

delivering a transmission ultrasound wave field from a transmission transducer array to a body part of a subject;

receiving transmission data from the transmission ultrasound wave field at a transmission receiver array;

delivering a reflection ultrasound wave field from a reflection transducer array to the body part of the subject;

receiving reflection data from the reflection ultrasound wave field at a reflection receiver array; and

generating an image of a bone or joint from at least one of the transmission data or the reflection data.

In another exemplary embodiment, the method can include generating the image of the bone or joint using transmission data.

In another exemplary embodiment, the method can further include:

generating speed of sound data from the transmission data; and

generating the image of the bone or joint from the speed of sound data.

In another exemplary embodiment, the method can further include refraction correcting the reflection data using the speed of sound data to generate a corrected reflection image of the bone or joint.

In another exemplary embodiment, the transmission ultrasound wave field is delivered prior to the reflection ultrasound wave field.

In another exemplary embodiment, the reflection ultrasound wave field is delivered prior to the transmission ultrasound wave field.

In another exemplary embodiment, the transmission ultrasound wave field and the reflection ultrasound wave field are delivered simultaneously.

In another exemplary embodiment, the method can further include:

repositioning at least one of the transmission transducer array or the reflection transducer array; and

repeating the steps of delivering the transmission ultrasound wave field, receiving the transmission data, delivering the reflection ultrasound wave field, and receiving the reflection data prior to generating the image of the bone or joint.

In another exemplary embodiment, the method can further include repeating the steps of the previous exemplary embodiment at a plurality of repositioned locations.

In another exemplary embodiment, the method can further include attaching a coupling device to the body part to facilitate transmission of ultrasound energy to and from the body part.

In another exemplary embodiment, the method can further include storing the transmission data and the reflection data in a nontransitory computer readable medium.

In another exemplary embodiment, the method can further include generating the image of the bone or joint using a computational processor functionally coupled to the nontransitory computer readable medium.

In another exemplary embodiment, the method can further include delivering a contrast agent to the body part.

In another exemplary embodiment, the contrast agent includes a member selected from the group consisting of microbubbles, microcapsules filled with air, microparticles containing biologic materials, antibodies, and molecular probes, including combinations thereof.

In another exemplary embodiment if the present disclosure, an ultrasound scanning system for imaging a bone or a joint of a subject comprises:

a transmission transducer array;

a transmission receiver array positioned to receive ultrasound energy transmitted through a body part and delivered from the transmission transducer array;

a reflection transducer array;

a reflection receiver array positioned to receive ultrasound energy reflected by the body part and delivered from the reflection transducer array; and

computation system functionally coupled to at least the transmission receiver array and to the reflection receiver array, and operable to generate an image of a bone or joint from at least one of transmission data received by the transmission receiver array or reflection data received by the reflection receiver array.

In another exemplary embodiment, the system includes a beam former functionally coupled to at least one of the transmission transducer array or the reflection transducer array.

In another exemplary embodiment, the computation system further comprises:

a nontransitory computer readable medium functionally coupled to the transmission receiver array and to the reflection receiver array, and operable to receive and store transmission data and reflection data; and

a computational module coupled to the nontransitory computer readable medium that is operable to generate the image of the bone or joint.

In another exemplary embodiment, the computational module is a computational processor.

In another exemplary embodiment, the computational module further comprises:

a plurality of interconnected nodes including at least one compute node and at least one data acquisition node;

wherein the at least one compute node includes a single board computer and a fibre channel host adaptor, and the at least one data acquisition node includes a single board computer, a fibre channel host adaptor, a waveform generator card, at least one data acquisition card, and at least one Mux card.

In another exemplary embodiment, the system further includes an imaging chamber functionally associated with the transmission transducer array, the transmission receiver array, the reflection transducer array, and the reflection receiver array, the imaging chamber being operable to receive and directly contact a coupler device operable to surround a bone or a joint of a subject.

In another exemplary embodiment, the system further includes a coupler device operable to engage within the imaging chamber and to surround a bone or a joint of a subject.

### EXAMPLES

### Example 1

### Human Knee Imaging

As shown in FIG. 8, an image of a human knee was obtained using QTUS imaging. To the right is shown the actual cut surface of the same human knee at the same level of the image, showing the synovium and cartilage in the coronal view.

### Example 2

### Muscle and Vasculature Imaging

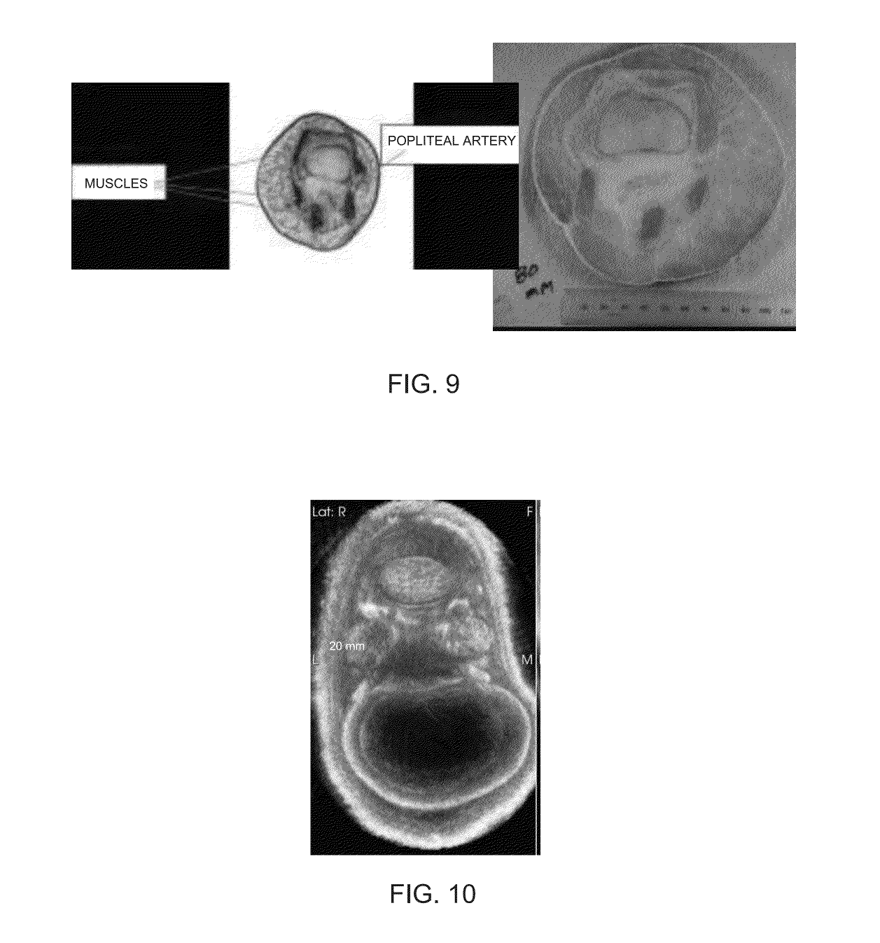

As is shown in FIG. 9, an image of a human knee was obtained using QTUS imaging. To the right is shown the actual cut surface of the same human knee at the same level of the image showing the muscles and blood vessels in the coronal view.

### Example 3

### Superficial and Deep Flexor Tendon Imaging

As is shown in FIG. 10, an image of a horse foreleg showing the superficial and deep flexor tendons in the coronal view was obtained using QTUS imaging.

### Example 4

### Flexor Tendon Imaging

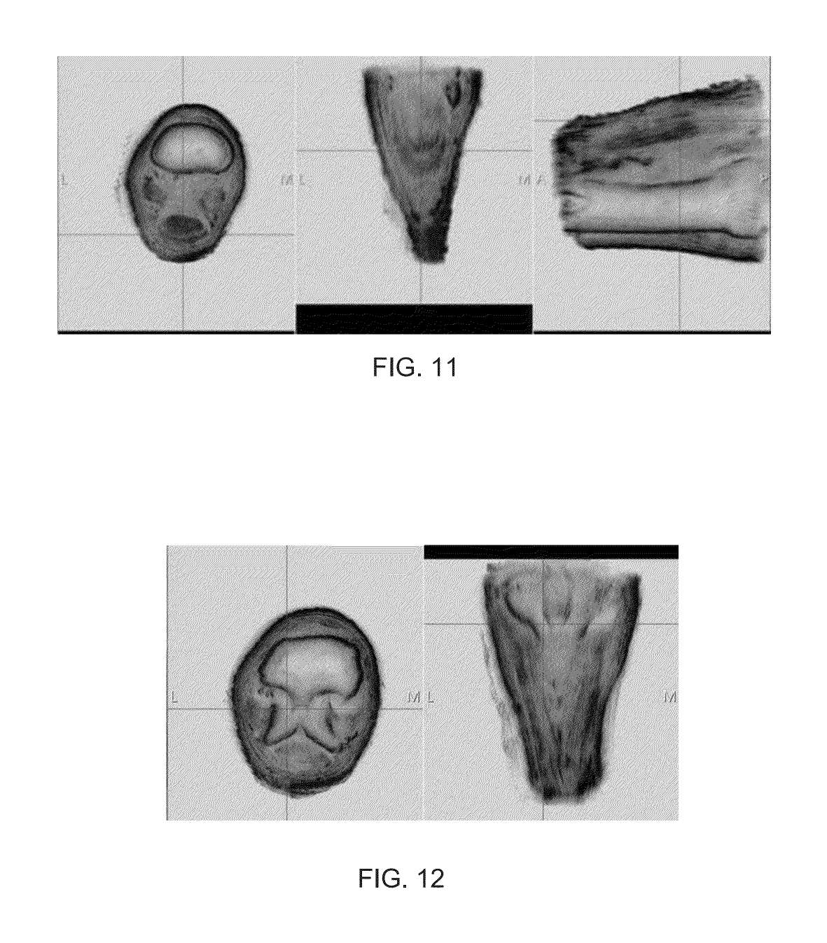

As is shown in FIG. 11, an image of a horse foreleg showing the superficial flexor tendon in the coronal, axial and sagittal views was obtained using QTUS imaging.

### Example 5

### Sesamoidean Ligament Imaging

As is shown in FIG. 12, an image of a horse foreleg showing the superficial flexor tendon in the coronal view and showing the sesamoid ligaments was obtained using QTUS imaging.

It is to be understood that the above-described arrangements and examples are only illustrative of the application of the principles of the present disclosure. Numerous modifications and alternative arrangements may be devised by those skilled in the art without departing from the spirit and scope of the present disclosure, and the appended claims are intended to cover such modifications and arrangements. Thus, while the present disclosure has been described above with particularity and detail in connection with what is presently deemed to be the most practical embodiments of the disclosure, it will be apparent to those of ordinary skill in the art that numerous modifications, including, but not limited to, variations in size, materials, shape, form, function and manner of operation, assembly and use may be made without departing from the principles and concepts set forth herein.

What is claimed is:

1. A method for imaging a bone or a joint of a subject, comprising:
delivering a transmission ultrasound wave field from a transmission transducer array to a body part of a subject;
receiving transmission data from the transmission ultrasound wave field at a transmission receiver array;
delivering a reflection ultrasound wave field from a reflection transducer array to the body part of the subject;
receiving reflection data from the reflection ultrasound wave field at a reflection receiver array; and
generating an image of a bone or joint from at least one of the transmission data or the reflection data.

2. The method of claim 1, further comprising generating the image of the bone or joint using transmission data.

3. The method of claim 2, further comprising:
generating speed of sound data from the transmission data; and
generating the image of the bone or joint from the speed of sound data.

4. The method of claim 3, further comprising refraction correcting the reflection data using the speed of sound data to generate a corrected reflection image of the bone or joint.

5. The method of claim 1, wherein the transmission ultrasound wave field is delivered prior to the reflection ultrasound wave field.

6. The method of claim 1, wherein the reflection ultrasound wave field is delivered prior to the transmission ultrasound wave field.

7. The method of claim 1, wherein the transmission ultrasound wave field and the reflection ultrasound wave field are delivered simultaneously.

8. The method of claim 1, further comprising:
repositioning at least one of the transmission transducer array or the reflection transducer array; and
repeating the steps of delivering the transmission ultrasound wave field, receiving the transmission data, delivering the reflection ultrasound wave field, and receiving the reflection data prior to generating the image of the bone or joint.

9. The method of claim 6, further comprising repeating the steps of claim 8 at a plurality of repositioned locations.

10. The method of claim 1, further comprising attaching a coupling device to the body part to facilitate transmission of ultrasound energy to and from the body part.

11. The method of claim 1, further comprising storing the transmission data and the reflection data in a nontransitory computer readable medium.

12. The method of claim 11, further comprising generating the image of the bone or joint using a computational processor functionally coupled to the nontransitory computer readable medium.

13. The method of claim 1, further comprising delivering a contrast agent to the body part.

14. The method of claim 13, wherein the contrast agent includes a member selected from the group consisting of microbubbles, microcapsules filled with air, microparticles containing biologic materials, antibodies, and molecular probes, including combinations thereof.

15. An ultrasound scanning system for imaging a bone or a joint of a subject, comprising:
a transmission transducer array;
a transmission receiver array positioned to receive ultrasound energy transmitted through a body part and delivered from the transmission transducer array;
a reflection transducer array;
a reflection receiver array positioned to receive ultrasound energy reflected by the body part and delivered from the reflection transducer array; and
computation system functionally coupled to at least the transmission receiver array and to the reflection receiver array, and operable to generate an image of a bone or joint from at least one of transmission data received by the transmission receiver array or reflection data received by the reflection receiver array.

16. The system of claim 14, further comprising a beam former functionally coupled to at least one of the transmission transducer array or the reflection transducer array.

17. The system of claim 14, where in the computation system further comprises:
a nontransitory computer readable medium functionally coupled to the transmission receiver array and to the reflection receiver array, and operable to receive and store transmission data and reflection data; and
a computational module coupled to the nontransitory computer readable medium that is operable to generate the image of the bone or joint.

18. The system of claim 17, wherein the computational module is a computational processor.

19. The system of claim 18, wherein the computational module further comprises:
a plurality of interconnected nodes including at least one compute node and at least one data acquisition node;
wherein the at least one compute node includes a single board computer and a fibre channel host adaptor, and the at least one data acquisition node includes a single board computer, a fibre channel host adaptor, a waveform generator card, at least one data acquisition card, and at least one Mux card.

20. The system of claim 15, further comprising an imaging chamber functionally associated with the transmission transducer array, the transmission receiver array, the reflection transducer array, and the reflection receiver array, the imaging chamber being operable to receive and directly contact a coupler device operable to surround a bone or a joint of a subject.

21. The system of claim 20, further comprising a coupler device operable to engage within the imaging chamber and to surround a bone or a joint of a subject.
